(19) United States
(12) Patent Application Publication
(10) Pub. No.: US 2025/0213292 A1
(43) Pub. Date: Jul. 3, 2025

(54) A SYSTEM FOR FORMING A FISTULA

(71) Applicant: CLEARSTREAM TECHNOLOGIES LIMITED, Enniscorthy, County Wexford (IE)

(72) Inventors: Andy Moll, Enniscorthy, County Wexford (IE); Olivia Palmer, Enniscorthy, County Wexford (IE); Michael Whelan, Enniscorthy, County Wexford (IE); John O'Shea, Enniscorthy, County Wexford (IE); Peter Akerele-ale, Enniscorthy, County Wexford (IE)

(21) Appl. No.: 18/847,478

(22) PCT Filed: Mar. 15, 2022
(86) PCT No.: PCT/EP2022/056648

§ 371 Date: Sep. 16, 2024

(51) Int. Cl. A61B 18/14; A61B 18/00; A61B 18/04; A61M 27/00

(52) U.S. Cl. CPC A61B 18/1492; A61M 27/002; A61B 2018/00184; A61B 2018/00404; A61B 2018/00601; A61B 18/042

(57) ABSTRACT

A system for forming a fistula between two vessels. The system comprises a first catheter comprising a housing with an opening, and an electrode disposed at least partially within the housing. The electrode comprises a distal portion, a proximal portion and an intermediate portion therebetween for contacting a vessel wall and forming the fistula. The first catheter is configured to provide resistance against lateral movement of the intermediate portion, for example, through a spring, a push element, a guide element or a pull wire.

### TECHNICAL FIELD

The present disclosure relates to a system for forming a fistula between two vessels.

### BACKGROUND

A fistula denotes a passageway formed between two internal organs. Forming a fistula between two blood vessels can have one or more functions. For example, the formation of a fistula between an artery and a vein may provide access to the vasculature for haemodialysis patients. Specifically, forming a fistula between an artery and a vein allows blood to flow quickly between the vessels while bypassing the capillaries. Needles, catheters, or other cannulas may then be inserted into the blood vessels near the fistula to draw blood from the circulatory system, pass it through a dialysis machine, and return it to the body. The quickened flow provided by the fistula may provide for effective haemodialysis.

In other instances, a fistula may be formed between two veins to form a veno-venous fistula. Such a veno-venous fistula may be used to treat portal venous hypertension. Specifically, cirrhosis or other liver diseases may cause increased resistance to flow from the portal veins draining from the intestine to the liver. This increased resistance may cause massive dilation of blood vessels, which may rupture spontaneously. To help prevent this, a fistula may be formed between a portal vein and one of the major branches, thereby lowering venous pressure in the portal vein.

US 2012/0302935 A1 and US 2017/0202616 A1 disclose systems and methods for forming a fistula using an endovascular approach. These systems comprise a first catheter having a housing with an electrode supplied with RF energy and a second catheter having a housing with a backstop. However, these systems can be limited in thicker calcified vessels by the distance the electrode can fire through the venous/arterial walls. When firing through thicker, calcified vessels, the RF energy supplied to the electrode must be at a higher power or supplied for a longer time to allow the electrode to cut through the thicker vessel. Because the electrode is in the form of a thin wire, a large amount of heat builds up in the electrode causing deformation of the electrode shape and resulting in a drop in the electrode height of up to 60%. The electrode may heat-set with this reduced height and, as a result, the electrode catheter cannot be reused.

In view of the above, there is a need in the art for a new catheter system which can more effectively form a fistula in calcified vessels.

### SUMMARY

In a first aspect of the present disclosure, there is provided a system for forming a fistula between two vessels. The system comprises a first catheter comprising a housing with an opening, and an electrode disposed at least partially within the housing. The electrode comprises a distal portion, a proximal portion and an intermediate portion therebetween for contacting a vessel wall and forming the fistula. The first catheter further comprises a spring attached to at least part of the electrode. The spring is configured to provide resistance against lateral movement of the intermediate portion.

In some embodiments, this may result in a system which can more easily and effectively form a fistula in calcified vessels.

Throughout this disclosure the term ‘fistula’ is used to denote a connection or passageway.

The electrode may have a radially expanded configuration and a radially contracted configuration.

Throughout this disclosure, the term “radially expanded configuration” refers to a configuration of the electrode where the electrode has a greater radial extent than in a “radially contracted configuration”. For example, in the radially expanded configuration, the intermediate portion may extend a first distance from the housing and in the radially contracted configuration, the intermediate portion may extend a second distance from the housing, where the first distance is greater than the second distance.

In some embodiments, this may allow the profile of the first catheter to be reduced for easier movement through a vessel.

In some embodiments, this may allow the electrode to be expanded and result in better fistula formation.

The spring may be configured to provide resistance against any force causing radial inward movement of the intermediate portion.

The electrode may comprise a leaf spring.

In some embodiments, this may allow the electrode to be bent and flexed without breaking.

Throughout this disclosure, the term ‘leaf spring’ is used to refer to a flexible curved strip of material which can be bent but will regain its original shape when released.

The electrode may have a convex shape.

In some embodiments, this may result in better fistula formation.

The electrode may be a ribbon wire.

In some embodiments, this may result in better fistula formation.

The spring may be at least partially positioned within the housing.

The proximal end of the electrode may be fixed with respect to the housing. The distal end of the electrode may be moveable with respect to the housing.

In some embodiments, this may allow the electrode to more easily move between the radially contracted configuration and the radially expanded configuration.

The spring may be attached to the distal portion of the electrode.

In some embodiments, this may provide resistance to lateral movement of the intermediate portion, and specifically radially inward movement of the intermediate portion.

The spring may be positioned between the distal end of the electrode and the distal end of the housing.

In some embodiments, this may provide resistance to lateral movement of the intermediate portion, and specifically radially inward movement of the intermediate portion.

The spring may comprise a helical spring or coil spring.

The helical spring may be a compression spring.

The spring may be attached to the intermediate portion of the electrode.

In some embodiments, this may provide resistance to lateral movement of the intermediate portion, and specifically radially inward movement of the intermediate portion.

The spring may be disposed between the housing and the intermediate portion.

In some embodiments, this may provide resistance to lateral movement of the intermediate portion, and specifically radially inward movement of the intermediate portion.

The spring may comprise a leaf spring.

The leaf spring may have a concave shape relative to the housing.

The spring may comprise a compression spring.

The spring may be integral with the electrode.

In some embodiments, this may allow simpler manufacture of the electrode.

The spring may comprise a torsion spring which is connected to the proximal portion of the electrode.

The housing may be made, at least partly, from a ceramic material.

In some embodiments, this may allow the housing to better withstand the heat and plasma generated by the electrode.

The system may further comprise a second catheter comprising a second housing and a backstop for the electrode.

The first catheter and the second catheter may comprise one or more sets of magnets positioned to align the electrode with the backstop.

In some embodiments, this may provide a simple way to allow exact alignment of the electrode and the backstop.

The backstop may be a recessed backstop which has a complementary shape to the electrode.

In some embodiments, this may allow the electrode to better engage with the backstop and result in better fistula formation.

The backstop may have a concave shape.

The system may further comprise a radiofrequency generator for supplying radiofrequency power to the electrode.

In a second aspect of the present invention, there is provided a system for forming a fistula between two vessels.

The system comprises a first catheter. The first catheter comprises a housing with an opening, and an electrode disposed at least partially within the housing. The electrode comprises a distal end, a proximal end and an intermediate portion therebetween for contacting a vessel wall and forming the fistula. The first catheter further comprises a push element connected to the distal end of a shaft for engaging and pushing the intermediate portion at least partially in a radially outward direction to thereby provide resistance against lateral movement of the intermediate portion.

In some embodiments, this may result in a system which can more easily and effectively form a fistula in calcified vessels.

The electrode may have a radially expanded configuration and a radially contracted configuration.

In some embodiments, this may allow the profile of the first catheter to be reduced for easier movement through a vessel.

In some embodiments, this may allow the electrode to be expanded and result in better fistula formation.

The electrode may comprise a leaf spring.

In some embodiments, this may allow the electrode to be bent and flexed without breaking.

The electrode may have a convex shape.

In some embodiments, this may result in better fistula formation.

The electrode may be a ribbon wire.

In some embodiments, this may result in better fistula formation.

The proximal end of the electrode may be fixed with respect to the housing. The distal end of the electrode may be moveable with respect to the housing.

In some embodiments, this may allow the electrode to more easily move between the radially contracted configuration and the radially expanded configuration.

The intermediate portion may have a radial inner surface. The radial inner surface may face the opening in the housing when in the radially expanded configuration.

Distal movement of the shaft may cause the push element to move at least partially in a radially outward direction and engage with the radial inner surface of the intermediate portion of the electrode.

In some embodiments, this may result in the electrode being more resistant to lateral movement, and specifically radially inward movement, and thereby result in more effective fistula formation in calcified vessels.

The shaft may be at least partially positioned within the housing.

The shaft may be moveable in a longitudinal direction with respect to the first catheter.

The system may further comprise a guide element for guiding the push element in at least a partially radial direction.

In some embodiments, this may allow the push element to more easily engage the intermediate portion of the electrode.

The push element may have an engagement surface for engaging the radial inner surface of the intermediate portion of the electrode.

The engagement surface may have a complementary shape to the radial inner surface of the intermediate portion.

In some embodiments, this may allow the push element to better engage the electrode.

The engagement surface of the push element may have a convex shape.

The push element may be made, at least partly, from a ceramic material.

In some embodiments, this may allow the push element to better withstand the heat and plasma generated by the electrode.

The housing may be made, at least partly, from a ceramic material.

In some embodiment, this may make the housing more resistant to the heat and plasma generated by the electrode.

The system may further comprise a second catheter comprising a second housing and a backstop for the electrode.

The first catheter and the second catheter may comprise one or more sets of magnets positioned to align the electrode with the backstop.

In some embodiments, this may provide a simple way to allow exact alignment of the electrode and the backstop.

The backstop may be a recessed backstop which has a complementary shape to the electrode.

In some embodiments, this may allow the electrode to better engage with the backstop and result in better fistula formation.

The backstop may have a concave shape.

The system may further comprise a radiofrequency generator for supplying radiofrequency power to the electrode.

In a third aspect of the present invention, there is provided a system for forming a fistula between two vessels. The system comprises a first catheter. The first catheter comprises a housing having an opening, and an electrode disposed at least partially within the housing. The electrode comprises a distal portion, a proximal portion and an intermediate portion therebetween for contacting a vessel wall and forming the fistula. The first catheter further comprises a guide element positioned in the housing distally of the electrode, and a moveable shaft connected to the distal portion of the electrode. Distal movement of the moveable shaft causes the electrode to contact the guide element and thereby provide resistance against lateral movement of the intermediate portion.

In some embodiments, this may result in a system which can more easily and effectively form a fistula in calcified vessels.

The electrode may have a radially expanded configuration and a radially contracted configuration.

In some embodiments, this may allow the profile of the first catheter to be reduced for easier movement through a vessel.

In some embodiments, this may allow the electrode to be expanded and result in better fistula formation.

The electrode may comprise a leaf spring.

In some embodiments, this may allow the electrode to be bent and flexed without breaking.

The electrode may have a convex shape.

In some embodiments, this may result in better fistula formation.

The electrode may be a ribbon wire.

In some embodiments, this may result in better fistula formation.

The proximal portion of the electrode may be fixed with respect to the housing. The distal portion of the electrode may be moveable with respect to the housing.

In some embodiments, this may allow the electrode to more easily move between the radially contracted configuration and the radially expanded configuration.

The moveable shaft may be located at least partly within the housing.

The guide element may have an engagement surface for contacting the electrode.

The engagement surface of the guide element may have a shape complementary to the shape of the electrode.

In some embodiments, this may allow the electrode to better engage with the guide element.

The engagement surface of the guide element may be concave.

The proximal portion of the electrode may have a hole. The moveable shaft may pass through the hole in the proximal end of the electrode.

The housing may be made, at least partly, from a ceramic material.

In some embodiment, this may allow the housing to better withstand the heat and plasma generated by the electrode.

The system may further comprise a second catheter comprising a second housing and a backstop for the electrode.

The first catheter and the second catheter may comprise one or more sets of magnets positioned to align the electrode with the backstop.

In some embodiments, this may provide a simple way to allow exact alignment of the electrode and the backstop.

The backstop may be a recessed backstop which has a complementary shape to the electrode.

In some embodiments, this may allow electrode to better engage with the backstop and thereby result in better fistula formation.

The backstop may have a concave shape.

The system may further comprise a radiofrequency generator for supplying radiofrequency power to the electrode.

In a fourth aspect of the present invention, there is provided a system for forming a fistula between two vessels.

The system comprises a first catheter. The first catheter comprises a housing with an opening, and an electrode disposed at least partially within the housing. The electrode comprises a distal portion, a proximal portion and an intermediate portion therebetween for contacting a vessel wall and forming the fistula. The first catheter further comprises a pull wire attached to the electrode. Proximal movement of the pull wire provides resistance against lateral movement of the intermediate portion.

In some embodiments, this may result in a system which can more easily and effectively form a fistula in calcified vessels.

The system may further comprise a wire support element disposed proximally of the electrode. The wire support element may be in contact with the pull wire.

The wire support element may be positioned laterally towards the side of the opening of the housing and may cause the pull wire to deflect in the direction of the opening of the housing.

In some embodiments, this may change the angle at which the pull wire pulls the electrode and thereby result in providing better resistance against lateral movement, and specifically radially inward movement, of the electrode.

The wire support element may be a torsion spring.

The wire support element may be a bar, rod or roller.

The pull wire may be attached to the distal portion of the electrode.

The distal portion of the electrode may form a hook for attachment of the pull wire.

In some embodiments, this may allow the pull wire to be more securely attached to the electrode.

The pull wire may be attached to the proximal portion of the electrode.

The electrode may have a radially expanded configuration and a radially contracted configuration.

In some embodiments, this may allow the profile of the first catheter to be reduced for easier movement through a vessel.

In some embodiments, this may allow the electrode to be expanded and result in better fistula formation.

The electrode may comprise a leaf spring.

In some embodiments, this may allow the electrode to be bent and flexed without breaking.

The electrode may have a convex shape.

In some embodiments, this may result in better fistula formation.

The electrode may be a ribbon wire.

In some embodiments, this may result in better fistula formation.

The housing may be made, at least partly, from a ceramic material.

In some embodiments, this may allow the housing to better withstand the heat and plasma from the electrode.

The system may further comprise a second catheter comprising a second housing and a backstop for the electrode.

The backstop may be non-conductive.

The backstop may be made, at least partly, from a ceramic material.

In some embodiments, this may allow the backstop to better withstand the heat and plasma generated by the electrode.

The first catheter and the second catheter may comprise one or more sets of magnets positioned to align the electrode with the backstop.

In some embodiments, this may provide a simple way to allow exact alignment of the electrode and the backstop.

The backstop may be a recessed backstop which has a complementary shape to the electrode.

In some this may result in better engagement of the electrode with the backstop and thereby better fistula formation.

The backstop may have a concave shape.

The system may further comprise a radiofrequency generator for supplying radiofrequency power to the electrode.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows an example of a system for forming a fistula.

FIG. 2 shows a side view of the system of FIG. 1 disposed within a blood vessel system prior to forming a fistula.

FIGS. 3A and 3B show an embodiment of a first catheter according to the present disclosure.

FIG. 4 shows a side view of another embodiment of a first catheter according to the present disclosure.

FIGS. 5A and 5B show side views of another embodiment of a first catheter according to the present disclosure in a contracted and expanded configuration, respectively.

FIGS. 6A and 6B show side views of another embodiment of a first catheter according to the present disclosure in a contracted and expanded configuration, respectively.

FIG. 6C shows a perspective view of the first catheter of FIG. 6A.

FIG. 7 shows a side view of another embodiment of a first catheter.

FIG. 8 shows a side view of another embodiment of a first catheter.

FIGS. 9A and 9B show side views of another embodiment of a first catheter according to the present disclosure in a contracted and expanded configuration, respectively.

### DETAILED DESCRIPTION

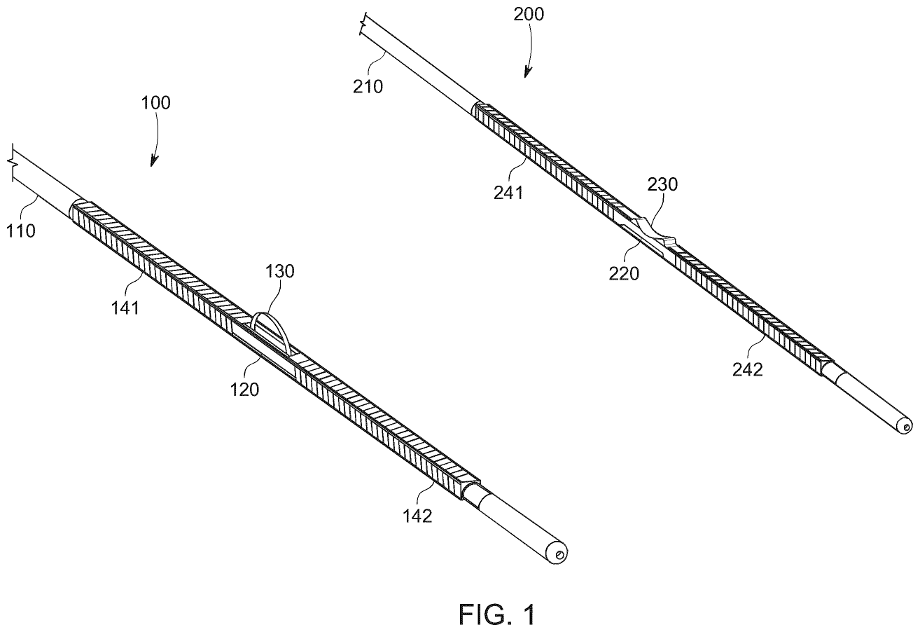

FIG. 1 shows an example of a catheter system for forming a fistula. The system comprises a first catheter 100 and a second catheter 200 which can be used together to form a fistula between two vessels.

The first catheter 100 comprises a catheter shaft 110 and a housing 120 disposed at the distal end of the shaft 110. The housing 120 has an opening and an electrode 130 which is partially disposed in the housing 120 and extends out of the opening. The housing 120 may be made from a non-conductive ceramic material which can withstand the heat and plasma generated by the electrode 130. The first catheter 100 also comprises a proximal set of magnets 141 and a distal set of magnets 142 which are disposed proximally and distally of the housing 120, respectively.

The second catheter 200 also comprises a catheter shaft 210 and a second housing 220 having a backstop 230 disposed at the distal end of the shaft 210. The backstop 230 has a concave portion which is shaped complimentary to the convex portion of the electrode 130. The second housing 220 and the backstop 230 may also be made from a non-conductive ceramic material to withstand the heat and plasma generated by the electrode 130. The second catheter 200 also comprises a proximal set of magnets 241, disposed proximally of the housing 220, and a distal set of magnets 242, disposed distally of the housing 220.

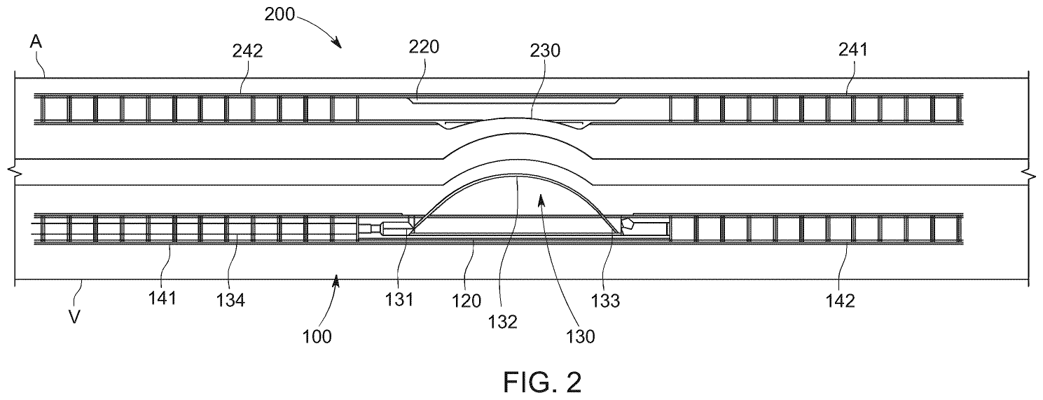

FIG. 2 shows the first catheter 100 disposed in a vein V and the second catheter 200 disposed in an adjacent artery A, prior to forming an arteriovenous fistula. As shown in FIG. 2, the electrode 130 comprises a proximal portion 131, an intermediate portion 132 and a distal portion 133. A connecting element 134 is connected to the proximal portion 131 and may extend along the shaft 110 of the first catheter 100. The proximal end of the connecting element 134 may be connected to an RF energy source, for example an ESU pencil, to allow RF energy to be supplied to the electrode 130. The proximal portion 131 may be fixed to the housing 120, for example, with a clamping mechanism or an adhesive. The intermediate portion 132 extends out of the opening of the housing 120 and comes into contact with the vessel wall for forming the fistula. The intermediate portion 132 may have a convex shape which extends away from the housing 120. The distance between the top of the intermediate portion 132 and the housing is the height of the electrode 130. The distal portion 133 is not fixed and can move longitudinally relative to the housing 120. This allows the electrode 130 to move between a radially contracted configuration and a radially expanded configuration. In the radially expanded configuration, the electrode 130 extends radially further from the housing 120 than in the radially contracted configuration.

The electrode 130 may be in the form of a ribbon wire and may be made from a number of suitable materials, such as one or more refractory metals. For example, the electrode 130 may comprise tungsten, molybdenum, niobium, tantalum, rhenium, or combinations and alloys thereof. The housing 120 may be made from a non-conductive ceramic material which can withstand the heat and plasma generated by the electrode 130.

In order to form a fistula between two vessels, such as artery A and vein V, the first catheter 100 may be introduced into the venous system through an access site and advanced to the treatment site where a fistula is to be formed. The second catheter 200 may be introduced into the arterial system through a second access site and also advanced to the treatment site where the fistula is to be formed. The first catheter 100 may be advanced to the treatment site inside a sheath. The sheath may compress the electrode 130 such that it is in the radially contracted configuration. This allows the first catheter 100 to more easily advance through the vessel due to the lower profile. The sheath may then be removed once the first catheter is in position at the treatment site which causes the electrode to move to the expanded position shown in FIG. 2.

The first catheter 100 and second catheter 200 may be advanced to the treatment site from opposite directions, as shown in FIG. 2, or alternatively, they may be advanced from the same direction.

Once the first catheter 100 and the second catheter 200 are positioned at the treatment site from opposite sides, the proximal set of magnets 141 of the first catheter 100 will be attracted to the distal set of magnets 242 of the second catheter 200 and align themselves with each other. Similarly, the distal set of magnets 142 of the first catheter 100 will be attracted to the proximal set of magnets 241 of the second catheter 200 and these sets of magnets will align with each other. This will result in the electrode 130 becoming aligned with the concave portion of the backstop 230. The backstop 230 is configured to compress the vessel walls in a localised region for ablation by the electrode 130 of the first catheter 100. The sets of magnets may also have the effect of pulling the artery A and vein V closer together.

Radiofrequency (RF) energy may then be supplied to the electrode 130 which causes the electrode 130 to heat up and generate a plasma. The plasma causes rapid dissociation of molecular bonds in organic compounds and allows the electrode 130 to cut through the venous and arterial vessel walls until it hits the backstop 230 to form the fistula.

However, the first catheter 100 is limited in the distance it can cut through the vessel walls. If the vessel walls are calcified, this can substantially increase the thickness of the vessel walls and may make it more difficult to cut through the vessel walls without deforming the electrode.

The catheter systems according to the present disclosure provide a solution to this problem.

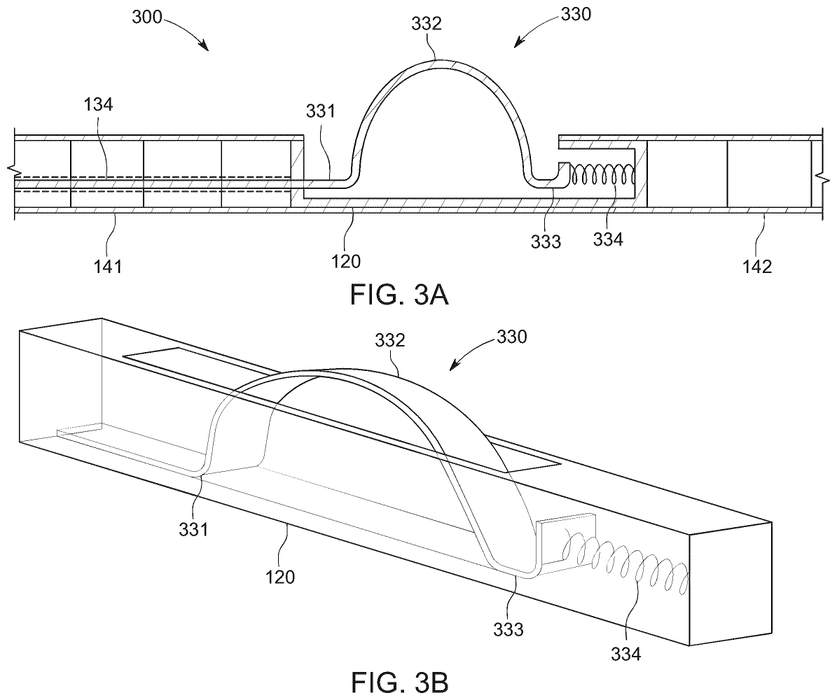

FIG. 3A shows a side view of a distal section of a first catheter 300 which is similar to first catheter 100. Throughout this disclosure, the same reference numerals are used to denote features which are identical across different embodiments.

Similar to first catheter 100, the first catheter 300 comprises an electrode 330 having a proximal portion 331, an intermediate portion 332 and a distal portion 333. The intermediate portion 332 has a convex shape and extends out of the opening of the housing 120 for contacting a vessel wall and forming a fistula. The proximal portion 331 may be fixed and coupled to a connecting element 134 which extends along the shaft of the catheter 300 and may be connected at its proximal end to an RF energy source. The electrode 330 may be made from the same materials as electrode 130 in FIG. 1.

The first catheter 300 further comprises a spring 334 which is connected to the distal portion 333 of the electrode 330. The spring 334 may be a helical compression spring and may be positioned between the distal portion 333 of the electrode 330 and the distal wall of the housing 120. The spring 334 provides resistance against distal movement of the distal portion 333 of the electrode 330 and thereby provides resistance against lateral movement, and particularly radial inward movement, of the intermediate portion 332. The spring 334 may be made from a number of suitable materials such as tungsten-rhenium or nitinol. The spring 334 may further comprise an insulating coating made from polyimide, for example. The spring 334 may also be insulated from the electrode 330 with an insulating material, such as polyimide, which may be positioned between the electrode 330 and the spring 334.

The first catheter 300 may be used together with second catheter 200 to form a fistula in the same manner as described for catheter 100 above. However, when the electrode 330 of first catheter 300 is pressing against the vessel wall, it can press against the wall with a greater force due to the increased resistance to lateral movement, and particularly radial inward movement, of the electrode 330.

This allows the first catheter 300 to more effectively cut through thicker calcified vessels, as the height of the electrode 330 does not drop significantly during the fistula forming process. Furthermore, under sufficient force, the spring 334 still allows longitudinal movement of the distal portion 333 such that the electrode 330 can be moved between the radially contracted and expanded configurations. This allows the first catheter 300 to be placed within a sheath and effectively delivered to the treatment site in the radially contracted configuration and then, in the radially expanded configuration when the sheath is removed, allows for a more effective fistula formation.

FIG. 3B shows a perspective view of the housing 120 and the electrode 330 of the first catheter 300. FIG. 3B illustrates the ribbon wire shape of the electrode 330 and shows how the spring 334 is positioned between the distal portion 333 of the electrode 330 and the distal wall of the housing 120.

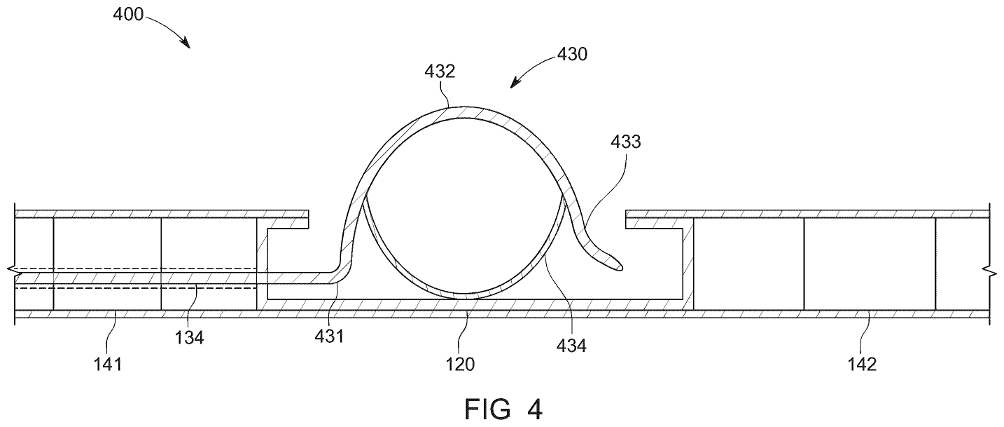

FIG. 4 shows another embodiment of a first catheter 400. The first catheter 400 is similar to first catheter 100 and the same reference numerals are used to denote identical features.

First catheter 400 comprises an electrode 430 having a proximal portion 431, an intermediate portion 432 and distal portion 433. The proximal portion 431 may be fixed and may connect to a connecting element 134. The intermediate portion 432 may have a convex shape and may extend out of the housing 120 to contact a vessel wall and form a fistula. The distal portion 433 may be free to move longitudinally within the housing 120, to allow the electrode 430 to move between the radially contracted configuration and the radially expanded configuration. The electrode 430 may have the same shape and may be made from the same material as electrode 130 of first catheter 100.

The first catheter 400 further comprises a spring 434 positioned between the intermediate portion 432 and the housing 120. The spring 434 may be a leaf spring having a concave shape with respect to the housing 120. The spring 434 may be made from the same materials as the electrode 430 and may be integrally formed with the electrode 430. For example, the spring 434 may be made from tungsten-rhenium or nitinol.

The spring 434 may also comprise an insulating coating made from polyimide, for example. In embodiments, the spring 434 may also be formed separately from the electrode 430 and separated from the electrode 430 with an insulator such as polyimide. The ends of the leaf spring 434 may be fixed to the intermediate portion 432 of the electrode 430 and the middle of the concave spring 434 may be in contact with the wall of the housing 120. The spring 434 may therefore provide resistance against lateral movement, and specifically radially inward movement, of the intermediate portion 432 whilst still allowing the electrode 430 to move between the radially contracted configuration and the radially expanded configuration.

The first catheter 400 may be used together with second catheter 200 to form a fistula in the same manner as described with respect to FIG. 2 above. Similar to the electrode 330 of first catheter 300 (FIGS. 3A and 3B), the electrode 430 can provide an increased force against the vessel wall without the height of the electrode 430 being reduced and may therefore allow the electrode 430 to more effectively cut through thicker calcified vessels.

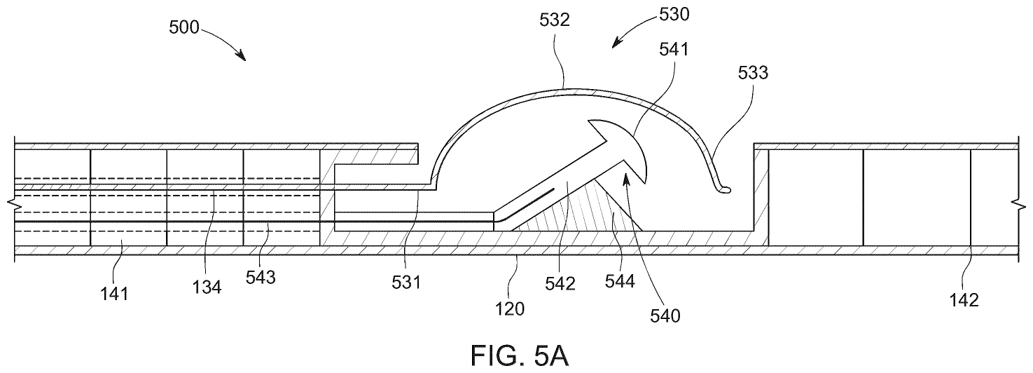

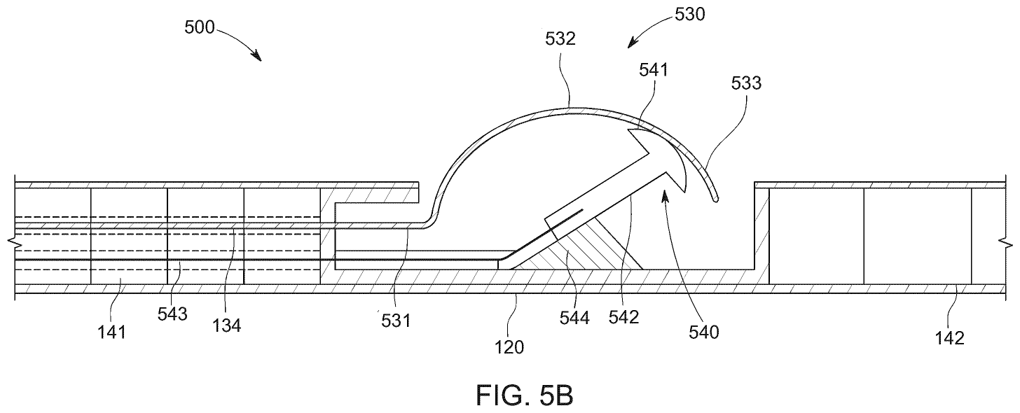

FIG. 5A shows another embodiment of a first catheter 500. The same reference numerals are used to denote identical features. The first catheter 500 comprises an electrode 530 having a proximal portion 531, intermediate portion 532 and distal portion 533. The proximal portion 531 may be fixed and may connect to a connecting element 134. The intermediate portion 532 may have a convex shape and extend out of the housing 120 for contacting a vessel wall and forming a fistula. The distal portion 533 may be free to move within the housing 120. The electrode 530 may therefore be able to move between a radially contracted configuration, as shown in FIG. 5A, and a radially expanded configuration, as shown in FIG. 5B. The electrode 530 may have the same shape and may be made from the same material as electrode 130 of first catheter 100.

The first catheter 500 further comprises a push element 540 connected to the distal end of a longitudinally moveable shaft 543. The push element 540 may have a convex engagement surface 541 for engaging with a radial inner surface of the intermediate portion 532 of the electrode 530. The push element 540 may further have a stem portion 542 which connects to the moveable shaft 543. A guide element 544 may be disposed within the housing 120 and may have a surface angled at least partially radially outward with respect to the longitudinal axis of the first catheter 500. The angle of the surface of the guide element 544 with respect to the longitudinal axis may be in the range of 20 to 90 degrees. In embodiments, the angle of the surface of the guide element 544 with respect to the longitudinal axis may be in the range of 40 to 70 degrees. The guide element 544 may be designed to guide the stem portion 542 of the push element 540 at least partially in a radially outward direction such that the engagement surface 541 can engage the intermediate portion 532 of the electrode 530. The push element 540 may be made from a non-conductive ceramic material to allow it to withstand the heat and plasma generated by the electrode 530 and prevent any arcing between the electrode 530 and the push element 540.

FIG. 5A shows the electrode 530 in the radially contracted configuration where the push element 540 is retracted and not in contact with the intermediate portion 532 of the electrode 530.

FIG. 5B shows the first catheter 500 with the electrode 530 in the radially expanded configuration.

The first catheter 500 may be used with second catheter 200 to form a fistula, similar to the method described with respect to FIG. 2 above. The first catheter 500 and the second catheter 200 may be introduced into respective vessels and advanced to the treatment site where a fistula between the two vessels is to be formed. When the first catheter 500 is introduced into the vessel and advanced to the treatment site, the electrode 530 may be in the radially contracted configuration shown in FIG. 5A. Furthermore, a sheath may be disposed around the first catheter 500 during introduction and advancement. When the first catheter 500 and second catheter 200 are positioned at the treatment site, the sheath may be removed and the catheters align due to the attraction of the magnets 141, 142 and 241, 242.

A user may then push the shaft 543 in a distal direction. Since the stem portion 542 of the push element 540 is in contact with the angled guide element 544 and connected to the shaft 543, distal movement of the shaft 543 will result in the push element 540 moving at least partly in a radially outward direction. The distal engagement surface 541 of the push element 540 comes into contact with the radial inner surface of the intermediate portion 532. If the shaft 543 is moved further distally, then the push element 540 will push the electrode 530 radially outward, thereby moving it from the radially contracted configuration to the radially expanded configuration, shown in FIG. 5B. RF energy may then be supplied to the electrode 530 to form the fistula.

The engagement of the push element 540 with the electrode 530 results in an increased resistance to lateral movement, specifically radially inward movement, of the electrode 530. The electrode 530 can therefore provide an increased force against the vessel wall without the height of the electrode 530 being reduced, as the push element 540 provides a counter-force. Hence the electrode 530 can cut through thicker calcified vessels more effectively. The first catheter 500 further allows the electrode 530 to move between the radially contracted and expanded configurations and the amount of expansion can even be controlled through the distance that the push element 540 is moved.

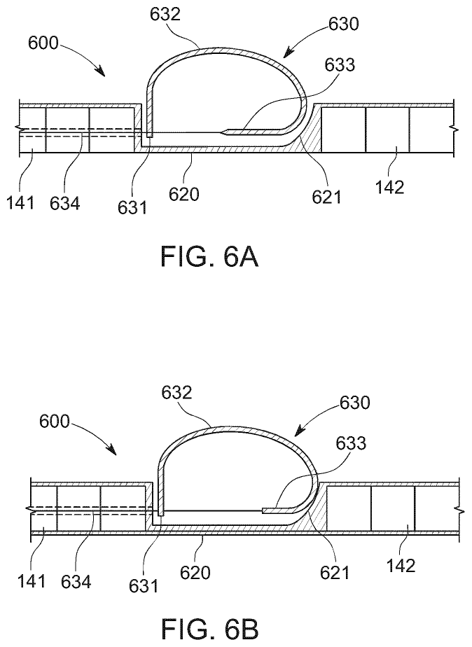

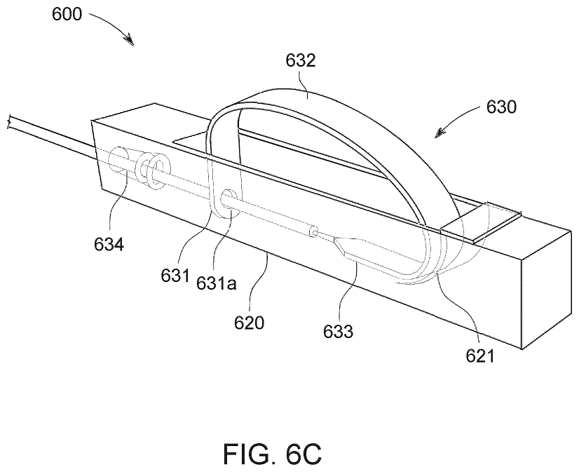

FIG. 6A shows another alternative embodiment of a first catheter 600. The same reference numerals are used to denote identical features.

The first catheter 600 comprises a housing 620 and an electrode 630 disposed at least partly within the housing 620. The housing 620 is similar to the housing 120 of first catheter 100 and may be made from the same materials. However, housing 620 further comprises a guide element 621. The guide element 621 may be disposed at the distal end of the housing 620 and may have a curved surface for engaging with the electrode 630.

The electrode 630 comprises a proximal portion 631, an intermediate portion 632 which may have a convex shape and extend from the housing 620 to contact a vessel wall, and a distal portion 633. The electrode 630 may be a ribbon wire and made from the same materials as electrode 130 of first catheter 100. The distal portion 633 of the electrode 630 may be bent so that the distal end 633 of the electrode faces in a proximal direction. A connecting element in the form of a moveable shaft 634 may be connected to the distal portion 633. The moveable shaft 634 may extend along the length of the shaft of the first catheter 600 and may connect, at its proximal end, to an RF energy source to allow the electrode 630 to be supplied with RF energy. The proximal portion 631 of the electrode 630 may be fixed to the housing 620 and may have a hole 631a (FIG. 6C) through which the moveable shaft 634 passes. The moveable shaft 634 is longitudinally moveable which allows the electrode 630 to be moved between a radially contracted configuration, illustrated in FIG. 6A, and a radially expanded configuration, as illustrated in FIG. 6B. FIG. 6B shows the electrode 630 of the first catheter 600 in the radially expanded configuration. In order to move the electrode 630 from the radially contracted configuration to the radially expanded configuration, a user may push the moveable shaft 634 in a distal direction. This will cause the distal portion 633 of the electrode 630 to move distally and engage the guide element 621. Further distal movement will result in the electrode 630 being pushed radially outward due to the curved shape of the guide element 621.

First catheter 600 may be used together with the second catheter 200 to form a fistula between two vessels in a similar manner as described with respect to FIG. 2 above. The first catheter 600 may be introduced into a vessel in the radially contracted configuration, with or without a sheath, and advanced to the treatment site. The second catheter 200 may be introduced into a second vessel and advanced to the treatment site. At the treatment site, attraction of the magnets 141, 142 of the first catheter 600 and the magnets 241, 242 of the second catheter may cause the electrode 630 to align with the backstop 230. The electrode 630 may then be moved from the radially contracted configuration to the radially expanded configuration by pushing the moveable shaft 634 distally such that the electrode 630 engages the guide element 621. RF energy may then be supplied to the electrode 630 through moveable shaft 634 to form the fistula between the vessels.

The engagement of the electrode 630 with the guide element 621 increases the height of the electrode and results in an increased resistance to lateral movement, specifically radially inward movement, of the electrode 630. The electrode 630 can therefore provide an increased force against the vessel wall without the height of the electrode being reduced and hence allows the electrode 630 to more effectively cut through thicker calcified vessels. The first catheter 600 further allows the amount of expansion of the electrode 630 to be controlled through movement of the moveable shaft 634.

FIG. 6C shows a perspective view of the housing 620 and the electrode 630 of first catheter 600. FIG. 6C illustrates the ribbon wire shape of the electrode 630 and shows the hole 631a in the proximal portion 631 of the electrode through which the moveable shaft 634 passes.

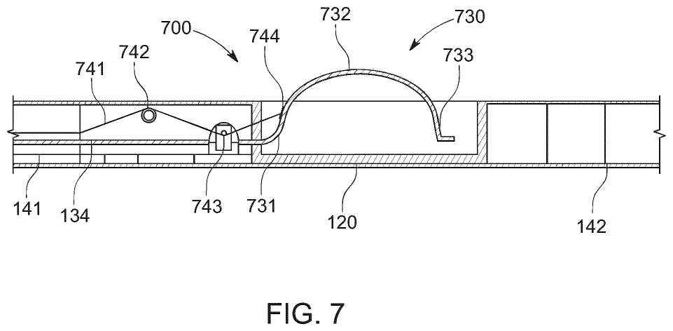

FIG. 7 shows another alternative embodiment of a first catheter 700. The same reference numerals are used to denote identical features.

The first catheter 700 comprises a housing 120 having an opening, and an electrode 730 having a proximal portion 731, an intermediate portion 732 and distal portion 733. The proximal portion 731 may be fixed with a clamping mechanism or an adhesive, for example. A connecting element 134 may be connected to the proximal portion 731 and may extend along the length of the shaft of the first catheter 700. The proximal end of the connecting element 134 may be connected to an RF energy source for supplying RF energy to the electrode 730. The intermediate portion 732 may have a convex shape and extend out of the opening of the housing 120 for contacting a vessel wall and forming a fistula. The distal portion 733 may be moveable within the housing 120 to allow the electrode to move from a radially contracted configuration to a radially expanded configuration. The electrode 730 may be in the form of a ribbon wire and may be made from the same materials as electrode 130 of first catheter 100.

The first catheter 700 further comprises a pull wire 741 which is attached to the proximal portion 731 of the electrode 730 at an attachment point 744. The pull wire 741 may be attached to the electrode 730 with an adhesive or a clamping mechanism, for example. The first catheter 700 may further comprise a first wire support element 742 which is in contact with the pull wire 741. The first catheter may also comprise an optional second wire support element 743, which is disposed distally of the first wire support element 742 and also in contact with the pull wire 741.

The first and second wire support elements 742, 743 may be, for example, a torsion spring, a bar, a rod or a roller. The first wire support element 742 may be positioned laterally offset from the center of the first catheter 700 towards an opening of the housing 120. The second wire support 743 element may be laterally offset from the center of the first catheter away from the opening of the housing 120. The first and second wire support elements 742, 743 change the angle at which the pull wire 741 attaches to the electrode 730 and thereby allow the height of the electrode 730 to be increased when the pull wire 741 is pulled proximally.

The first catheter 700 may be used together with the second catheter 200 to form a fistula in a similar manner as described above with respect to FIG. 2. Once the first catheter 700 and the second catheter 200 are positioned at the treatment site and aligned through attraction of the magnets 141, 142 and 241, 242, a user can pull the pull wire 741 in a proximal direction. This will result in an increased resistance to lateral movement, specifically radially inward movement, of the electrode 730 as the pull wire provides an opposing force and prevents the electrode 730 from being pushed down. Proximal movement of the pull wire 741 may also increase the height of the electrode 730. The electrode 730 can therefore provide an increased force against the vessel wall without the height of the electrode being reduced and hence allows the electrode 730 to more effectively cut through thicker calcified vessels.

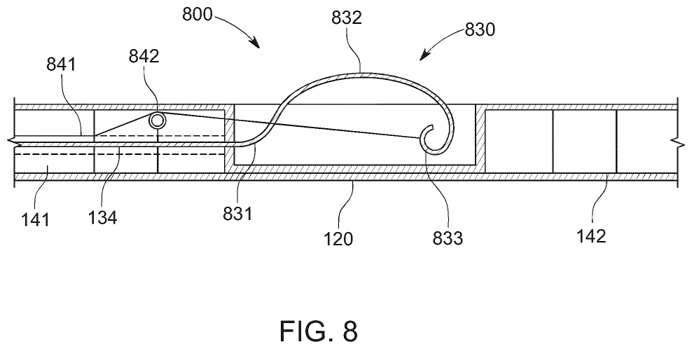

FIG. 8 shows another alternative embodiment of a first catheter 800. The same reference numerals are used to denote identical features of previous embodiments.

The first catheter 800 comprises a housing 120 having an opening and an electrode 830 having a proximal portion 831, intermediate portion 832 and distal portion 833. The proximal portion 831 may be fixed with a clamping mechanism or an adhesive, for example. A connecting element 134 may be connected to the proximal portion 831 and extend along the length of the shaft 110 of the first catheter 800 with the proximal end of the connecting element 134 connected to an RF energy source. The intermediate portion 832 may have a convex shape extending out of the opening of the housing 120 for contacting a vessel wall and forming a fistula. The distal portion 833 may be free to move within the housing 120 to allow the electrode 830 to move between a radially contracted configuration to a radially expanded configuration. The electrode 830 may be in the forms of a ribbon wire and may be made from the same materials as electrode 130 of first catheter 100.

A pull wire 841 is attached to the distal portion 833 of the electrode 830. The distal portion 833 of the electrode 830 may be bent and formed into a hook to allow easier attachment of the pull wire 841. The pull wire 841 may be attached to the electrode 830 using an adhesive or a clamping mechanism, for example.

The first catheter 800 further comprises a wire support element 842 which may be disposed proximally of the electrode 830 and in contact with the pull wire 841. The wire support element 842 may be radially offset from the center of the first catheter 800 in a direction towards the opening of the housing 120. The wire support element 842 may change the angle at which the pull wire 841 pulls the distal portion 833 of the electrode 830 and thereby may allow the height of the electrode 830 to be increased when the pull wire 841 is pulled proximally.

The first catheter 800 may be used together with the second catheter 200 to form a fistula in a similar manner as described above with respect to FIG. 2. Once the first catheter 800 and the second catheter 200 are positioned at the treatment site and aligned through attraction of the magnets 141, 142 and 241, 242, a user can pull the pull wire 841 in a proximal direction. This will result in an increased resistance to lateral movement, specifically radially inward movement, of the electrode 830 as the pull wire 841 applies a bending force to the electrode 830. Proximal movement of the pull wire 841 may also increase the height of the electrode 830. The electrode 830 can therefore provide an increased force against the vessel wall without the height of the electrode being reduced and hence allows the electrode 830 to more effectively cut through thicker calcified vessels.

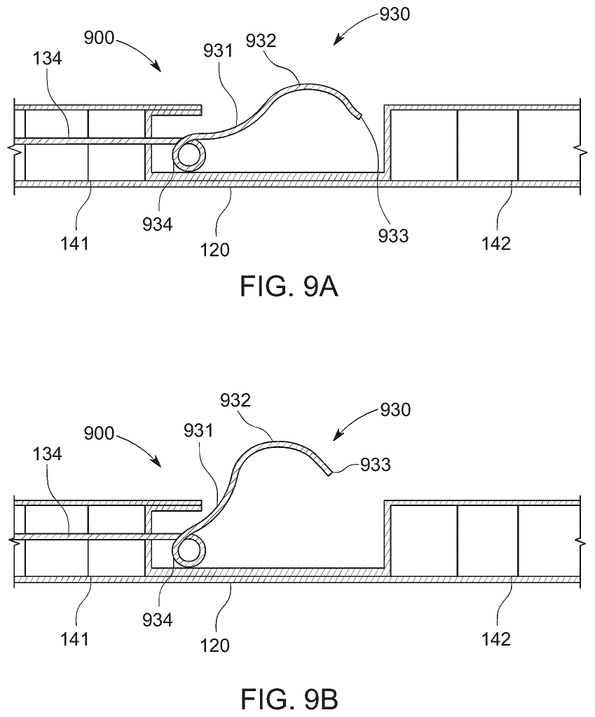

FIG. 9A shows another alternative embodiment of a first catheter 900 having a housing 120. The first catheter 900 comprises an electrode 930 having a proximal portion 931, intermediate portion 932 and distal portion 933. The proximal portion 931 is connected to a torsion spring 934 which may connect to a connecting element 134. An RF energy source may be connected to the distal end of connecting element 134 for supplying the electrode 930 with RF energy. The intermediate portion 932 may have a convex shape and extend out of the housing 120 for contacting a vessel wall and forming a fistula. The distal portion 933 may be free to move relative to the housing 120. The electrode 930 may therefore be able move between a radially contracted configuration, shown in FIG. 9A, and a radially expanded configuration. The electrode 930 may have the same shape and may be made from the same material as electrode 130 of first catheter 100.

The torsion spring 934 may be made from the same material as the electrode 930 and formed integrally with the electrode 930.

FIG. 9B shows the first catheter 900 with the electrode 930 in a radially expanded configuration. In the radially expanded configuration, the height of the electrode 930 is greater than in the radially contracted configuration of FIG. 9A. The torsion spring 934 provides a radially outward force on the electrode 930 which causes it to move from the radially contracted configuration to the radially expanded configuration. This radially outward force also provides the increased resistance to radially inward movement of the intermediate portion 932.

The first catheter 900 may be used with second catheter 200 to form a fistula, similar to the method described with respect to FIG. 2 above. The first catheter 900 may be introduced into a blood vessel inside a sheath which keeps the electrode 930 in the radially contracted configuration. Once the first catheter 900 is positioned at the treatment site, the sheath can be removed which will cause the electrode 930 to move from the radially contracted configuration to the radially expanded configuration.

The torsion spring 934 provides an increased resistance to lateral movement, specifically radially inward movement, of the electrode 930 as the torsion spring 934 pushes the electrode 930 radially outward. The electrode 930 can therefore provide an increased force against the vessel wall without the height of the electrode 930 being reduced and hence allows the electrode 930 to more effectively cut through thicker calcified vessels when RF energy is applied to the electrode 930.

Various modifications will be apparent to those skilled in the art.

The electrode of any of the embodiments may not be in the form of a ribbon wire but may have any other suitable shape, such as a circular or oval wire.

The intermediate portion of the electrode of any of the embodiments may not have a convex shape but may be any other suitable type of shape such as V-shaped or U-shaped.

The electrode of any of the embodiments is not limited to being made form a refractory material and may be made from any other suitable conductive type of material.

The first catheter may not comprise any magnets 141, 142.

The housing 120, 620 is not limited to a ceramic material and may be made form any other suitable type of material.

The second catheter may not comprise any magnets 241, 242.

The housing 220 of the second catheter 200 is not limited to being made from a ceramic material and may be made from any other type of suitable material.

The backstop 230 of the second catheter is not limited to a concave shape and may have any other type of suitable shape, such as V-shaped, U-shaped or rectangular-shaped, for example.

The spring 334 is not limited to a helical compression spring and may be any other suitable type of spring, such as a leaf spring, for example.

Similarly, the spring 434 is not limited to being a leaf spring and may be any type of other suitable spring, such as a helical compression spring.

The engagement surface 541 of the push element 540 is not limited to a convex surface and may have any suitable shape, such as, for example, a planar surface, a V-shaped surface or a hemispherical surface.

The guide element 544 may have a surface disposed at a range of angles to the longitudinal axis, for example 20 to 90 degrees, or 40 to 70 degrees.

The guide element 544 may have a curved surface rather than a straight surface.

The stem portion 542 of the push element 540 may be curved to engage with the curved surface of the guide element.

The guide element 621 is not limited to a curved shape and may be any other suitable shape.

The proximal portion 631 of electrode 630 may not comprise a hole. The moveable shaft 634 may, for example, extend below the proximal portion 631 of the electrode 630.

The first catheter 700 may only have one wire support element.

The first catheter 700 may have no wire support element.

The first catheter 800 may have no wire support element.

The distal portion 833 of the electrode 830 may not be hook-shaped.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A system for forming a fistula between two vessels comprising:
a first catheter comprising a housing with an opening;
an electrode disposed at least partially within the housing, the electrode comprising a distal portion, a proximal portion and an intermediate portion therebetween for contacting a vessel wall and forming the fistula; and
a spring attached to at least part of the electrode, and
wherein the spring is configured to provide resistance against lateral movement of the intermediate portion.

2. (canceled)

3. The system of claim 1, wherein the spring is configured to provide resistance against any force causing radial inward movement of the intermediate portion.

4-7. (canceled)

8. The system of claim 1, wherein the spring is attached to the distal portion of the electrode.

9-10. (canceled)

11. The system of claim 1, wherein the spring is attached to the intermediate portion of the electrode.

12. The system of claim 11, wherein the spring is disposed between the housing and the intermediate portion.

13. The system of claim 11, wherein the spring comprises a leaf spring.

14. The system of claim 11, wherein the spring is integral with the electrode.

15-17. (canceled)

18. A system for forming a fistula between two vessels comprising:
a first catheter comprising a housing with an opening;
an electrode disposed at least partially within the housing, the electrode comprising a distal end, a proximal end and an intermediate portion therebetween for contacting a vessel wall and forming the fistula; and
a push element connected to the distal end of a shaft for engaging and pushing the intermediate portion at least partially in a radially outward direction to thereby provide resistance against lateral movement of the intermediate portion.

19-22. (canceled)

23. The system of claim 18, wherein the intermediate portion has a radial inner surface.

24. The system of claim 23, wherein distal movement of the shaft causes the push element to move at least partially in a radially outward direction and engage with the radial inner surface of the intermediate portion of the electrode.

25. (canceled)

26. The system of claim 18, wherein the shaft is moveable in a longitudinal direction with respect to the first catheter.

27. The system of claim 18, further comprising a guide element for guiding the push element in at least a partially radial direction.

28-34. (canceled)

35. A system for forming a fistula between two vessels comprising:
a first catheter comprising a housing having an opening;
an electrode disposed at least partially within the housing, the electrode comprising a distal portion, a proximal portion and an intermediate portion therebetween for contacting a vessel wall and forming the fistula;
a guide element positioned in the housing distally of the electrode; and
a moveable shaft connected to the distal portion of the electrode,
wherein distal movement of the moveable shaft causes the electrode to contact the guide element and thereby provide resistance against lateral movement of the intermediate portion.

36-40. (canceled)

41. The system of claim 35, wherein the guide element has an engagement surface for contacting the electrode.

42. The system of claim 41, wherein the shape of the engagement surface of the guide element is complementary to the shape of the electrode.

43. (canceled)

44. The system of claim 35, wherein the proximal portion of the electrode has a hole, and wherein the moveable shaft passes through the hole in the proximal end of the electrode.

45-48. (canceled)

49. A system for forming a fistula between two vessels comprising:
a first catheter comprising a housing with an opening;
an electrode disposed at least partially within the housing, the electrode comprising a distal portion, a proximal portion and an intermediate portion therebetween for contacting a vessel wall and forming the fistula; and
a pull wire attached to the electrode, and
wherein proximal movement of the pull wire provides resistance against lateral movement of the intermediate portion.

50. The system of claim 49, further comprising a wire support element disposed proximally of the electrode, the wire support element being in contact with the pull wire.

51. The system of claim 50, wherein the wire support element is positioned laterally towards the side of the opening of the housing and causes the pull wire to deflect in the direction of the opening of the housing.

52. The system of claim 50, wherein the wire support element is a torsion spring, a bar, rod or roller.

53-64. (canceled)
